(19) United States
(12) Patent Application Publication
(10) Pub. No.: US 2011/0264080 A1
(43) Pub. Date: Oct. 27, 2011

(54) Medical Devices Having Extremely High Radiopacity Containing Ytterbium Compound

(75) Inventors: Hyung Sup Lim, Ansan (KR); Young Cheol Yoo, Ansan (KR); O Sung Kwon, Ansan (KR); Sang Min Kim, Ansan (KR); Gyung Man Kim, Ansan (KR)

(73) Assignee: Sukgyung AT Co., Ltd., Ansan (KR)

(21) Appl. No.: 13/093,470

(22) Filed: Apr. 25, 2011

Related U.S. Application Data
(60) Provisional application No. 61/327,162, filed on Apr. 23, 2010.

(51) Int. Cl. A61M 25/00; A61L 29/04; B29C 70/00; B29C 70/88; A61B 17/00; A61L 29/18; B82Y 5/00

(52) U.S. Cl. 606/1; 523/105; 264/241; 977/927

(57) ABSTRACT

A medical device, such as a catheter, exhibiting high radiopaque properties as well as optical transparency is disclosed. Further, radiopaque materials and process conditions to produce such a material as well as a medical device, such as a catheter, exhibiting high radiopaque and optically transparent properties are also disclosed.

### RELATED APPLICATIONS

This application claims priority to and incorporates by reference U.S. Provisional Application No. 61/327,162 filed Apr. 23, 2010. Further, U.S. Pat. Nos. 7,175,700, 6,971,391, 6,746,661, 6,306,926, 6,183,409, 6,159,141, 6,060,036, 5,417,959 and 4,647,447 and U.S. Published Application Number 2008/0145820 are hereby incorporated by reference.

### TECHNICAL FIELD OF THE INVENTION

The present invention relates to precision medical devices exhibiting high radiopaque and optical transparency, as well as processing conditions which produce a medical device exhibiting high radiopaque and optically transparent properties.

### BACKGROUND OF THE INVENTION

Generally, such a precision medical device, e.g., a catheter, is used in interventional techniques to perform diagnostic and therapeutic procedures, such as stenting and angioplasty. Thus, it is generally desirable that catheters be radiopaque because it is often necessary to determine the precise location of a catheter within its host by x-ray examination. In addition, it would be advantageous if catheters were optically transparent so that the flow of fluids there through could be observed.

Several types of catheters are made of a material which is radiopaque, so that the catheter is visible under fluoroscopy or other x-ray diagnosis. Typically, catheters for the arteriovenus system are made radiopaque generally by compounding into the plastic material of the catheter a radiopaque material. Suitable radiopaque materials that have been used include gold, tantalum, platinum, bismuth, iridium, zirconium, iodine, titanium, barium, silver, tin, alloys of these metals, and metal alloy compounds. Moreover, such radiopaque materials are used in submicron sizes as larger particles may compromise the structural integrity of a catheter, and are therefore inappropriate to provide radiopacity of a catheter used in certain medical applications.

However, difficulties arise with mixing a radiopaque material, typically in a powder or granular form with the plastic material of the catheter. One potential limitation of this approach is that due to the size of the powder granules of the radiopaque material, the inner and outer surfaces of the catheter may become rough or coarse. This may be particularly problematic when the concentration of the radiopaque filler material is high, especially near the surface. For some radiopaque filler materials, high concentrations may be required to achieve the desired x-ray visibility.

Another limitation may be that the radiopaque filler material may cause the plastic binder materials to lose their original and desired thermoplastic properties. Hard granular radiopaque materials in particular may detract from the desired flexibility ductility and maneuverability of the resulting tubing in direct proportion to the amount of radiopacity that they impart.

Other drawbacks of utilizing such radiopaque material include a loss of radiopacity in the distal end of the flexible tip. This may be due to a lack of cohesion with the distal tip portion of the catheter and creates a rupture risk of the thin-walled portion of the catheter. Because determining position of the catheter is typically critical to the success of most interventional procedures, there is a need for a catheter having an improved radiopacity and optically transparent properties to avoid the drawbacks of previously known designs.

Additional known drawbacks are encountered when a precision instrument such as a catheter is made using heavy metal compounds such as bismuth oxide, bismuth oxy-chloride, bismuth carbonate, barium sulfate, and tungsten to exhibit radiopacity within the catheter. Such materials have either yellow, pale yellow or black colors, resulting in a finished product that is neither sheer nor optically transparent when mixed with the plastic portion during the extruding process.

Moreover, white barium sulfate generally used to manufacture catheters is known to exhibit high refractive index properties, making it difficult to manufacture optically transparent catheter tubes. As will be understood to anyone skilled in the art, high refractive index results in low optical transparency, and vice versa.

In view of the foregoing, the present inventors have conducted extensive studies with a view toward developing an improved ytterbium compound for use in producing medical devices, which are not only effective for exhibiting high radiopacity and high optical transparency, as compared to the radiopacity and optical transparency achieved by conventional heavy metals, but also can be easily produced during the extruding process.

### SUMMARY OF THE INVENTION

Accordingly, it is an object of the present invention to provide a radiopaque medical device which is manufactured using a material containing ytterbium nanoparticles. As contemplated herein, such ytterbium nanoparticles may be incorporated into and dispersed within the matrix of a polymer without substantially affecting the mechanical properties of the polymer.

Methods of manufacturing the medical device of the present invention are also provided wherein radiopaque nanoparticles of ytterbium are obtained and added to a polymer that has been heated above its melting point. The mixture is then agitated to uniformly disperse the nanoparticles. The polymer may then be processed in accordance with well-known molding techniques.

These and other aspects of the invention may be understood more readily from the following description and the appended drawings.

### BRIEF DESCRIPTION OF THE DRAWINGS

The invention may be better understood with reference to the appended drawings, for which a description of each is provided below. The components in the drawings are not necessarily to scale, emphasis instead being placed upon illustrating the principles of the invention. In the drawings, like reference numbers are intended to designate corresponding parts throughout the description.

FIG. 1 is a magnified photograph of the particle sizes of the ytterbium trioxide manufactured in accordance with the invention, as shown through the Field Emission Scanning Electron Microscope (FE-SEM).

FIG. 2 is a graph depicting the analysis of the crystal structure of the ytterbium trioxide, as shown in FIG. 1, manufactured in accordance with the invention.

FIG. 3 is a magnified photograph of the particle sizes of the SG-YBO series manufactured in accordance with the invention, as shown through the FE-SEM.

FIG. 4 is a graph depicting the analysis of the crystal structure of the SG-YBO series, as shown in FIG. 3, manufactured in accordance with the invention.

FIG. 5 is a magnified photograph of the particle sizes of the SG-YBF series (SG-YBF40-4-401) manufactured in accordance with the invention, as shown through the FE-SEM.

FIG. 6 is a graph depicting the analysis of the crystal structure of the SG-YBF series, as shown in FIG. 5, manufactured in accordance with the invention.

FIG. 7 is a magnified photograph of the particle sizes of another SG-YBF series (SG-YBF100) series manufactured in accordance with the invention, as shown through the FE-SEM.

FIG. 8 is a graph depicting the analysis of the crystal structure of the SG-YBF series, as shown in FIG. 7, manufactured in accordance with the invention.

FIG. 9 is a magnified photograph of the particle sizes of another SG-YBF series (SG-YBF100-702) manufactured in accordance with the invention, as shown through the FE-SEM.

FIG. 10 is a graph depicting the analysis of the crystal structure of the SG-YBF series, as shown in FIG. 9, manufactured in accordance with the invention.

FIG. 11 is a magnified photograph of the particle sizes of another SG-YBF series (SG-YBF250N) manufactured in accordance with the invention, as shown through the FE-SEM.

FIG. 12 is a graph depicting the analysis of the crystal structure of the SG-YBF series, as shown in FIG. 11, manufactured in accordance with the invention.

### DETAILED DESCRIPTION OF THE INVENTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. For example, the following discussion is specifically directed to a flexible, steerable catheter, though it should be understood that other medical devices may benefit from the advantages of the disclosed invention.

In a preferred embodiment, a flexible, steerable catheter comprises a plastic formulation containing material having higher radiopaque properties than prior art devices. The material is also colorless and optically transparent. Such properties are possible, in part, because the distal tip of the flexible, steerable catheter utilizes nanoparticles of ytterbium metal, alloys of ytterbium, and/or compounds of ytterbium. It has been determined that such a catheter containing ytterbium compounds exhibits four times (4×) higher radiopacity than other conventional heavy metals.

### EXAMPLES

In the following descriptions, illustrative methods of making a steerable catheter using nanoparticles of ytterbium trioxide are described.

### Example 1

Using known or acquired techniques, ytterbium trioxide (Yb₂O₃) in micron size is blended with an inorganic acid to produce composite ytterbium hydroxide and ytterbium carbonate. Suitable inorganic acids include, for example, nitric acid, hydrogen chloride, and sulfuric acid. Generally, the mixture may also produce therefrom, for example, ammonium hydroxide (NH₄OH), sodium hydroxide (NaOH), potassium hydroxide (KOH), ammonium carbonate ((NH₄)₂CO₃), sodium carbonate (Na₂CO₃), and/or potassium carbonate (K₂CO₃). Thus, from the mixture generating ytterbium hydroxide and ytterbium carbonate, the aforementioned soluble compositions are removed and the resulting mixture is placed in a dryer and heated in a furnace to produce a purified nano sized ytterbium trioxide.

The resulting composition of ytterbium trioxide was determined to have an approximate 99.9% purity and the particle sizes ranged from about 30 nm to about 2 μm.

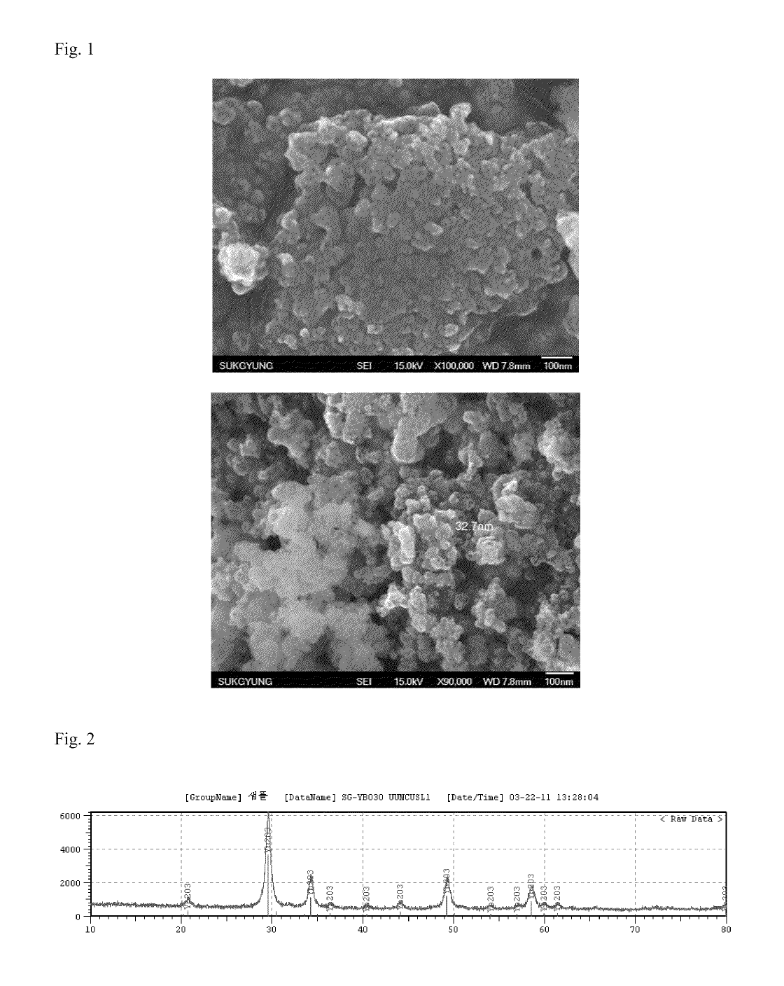

Utilizing Field Emission Scanning Electron Microscopy (FE-SEM), the actual particle sizes of the ytterbium trioxide manufactured in accordance with the invention are shown in FIG. 1. The specific surface area of the ytterbium trioxide was determined to be approximately 35 m²/g and the refractive index was measured to be 1.94. Hereinafter, the ytterbium trioxide is referenced as a radiopaque agent powder.

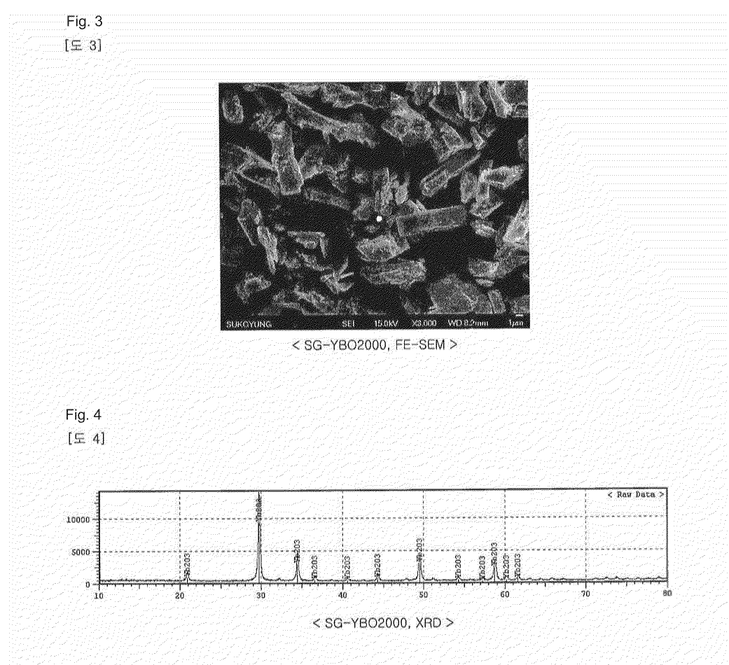

Further utilizing the FE-SEM, the actual particle sizes of the ytterbium trioxide manufactured in accordance with the invention are shown in FIG. 3. The specific surface area of the ytterbium trioxide was determined to be approximately 1 m²/g and the refractive index was measured to be 1.94.

It is preferred, but not necessary, that the radiopaque agent powder is blended with a plastic material to produce a material having a composition of about 30 parts nanoparticles of ytterbium trioxide, about 70 parts plastic material, and about 0.1 parts plasticizer, dispersant additives. Other composition ratios may be suitable for various uses and devices. Preferably, a polyvinyl chloride (PVC) resin is used as the suitable plastic material. As will be understood, other suitable plastic materials include, for example, silicones, polypropylene, polyester, polyolefin, fluoropolymers such as polytetrafluoroethylene (PTFE), polyethyl urethanes, polyethylene terephthalate (PET) and blends or mixtures thereof.

The radiopaque agent powder, selected for its uniform particle shape and controlled particle size distribution as described above, is subsequently introduced into a vat containing the molten plastic material. Thus, the solid powder, molten polymer and additives are homogenized as they are agitated and subsequently introduced into the melt stream via an extrusion process.

The described mixture composition was analyzed using an X-ray diffractometer wherein, as shown in FIGS. 2 and 4, the preferable composition of the present invention contains generally about 30 parts of ytterbium trioxide per 100 weight parts of the composition. [ignore the comment]

The resulting mixtures were then tested for radiopacity and appearance for translucency:

Radiopacity: 3.8 mm as Aluminum thickness.

Appearance: Translucent.

### Example 2

Using known or acquired techniques, first, commercial grade ytterbium trioxide (Yb₂0₃) in micron size is dissolved in inorganic acid. Suitable inorganic acids include, for example, nitric acid, hydrogen chloride, and sulfuric acid. The mixture is then dissolved in a solution of alkaline carbonate compounds, such as ammonium carbonate, sodium carbonate, and/or potassium carbonate to produce ytterbium carbonate. Thereafter, impurities are removed from the solution by way of known techniques, wherein the solution further mixed with hydrogen fluoride. From the mixture, the resulting amorphous ytterbium fluoride was obtained and was placed in a dryer and then heated in a furnace to produce a purified nano sized ytterbium fluoride composition. The resulting ytterbium fluoride was determined to have an approximate 99.9% purity and the particle sizes ranged from about 40 nm to about 250 nm. The specific surface area of the ytterbium fluoride was determined to be approximately 18 m²/g and the refractive index was measured to be 1.94.

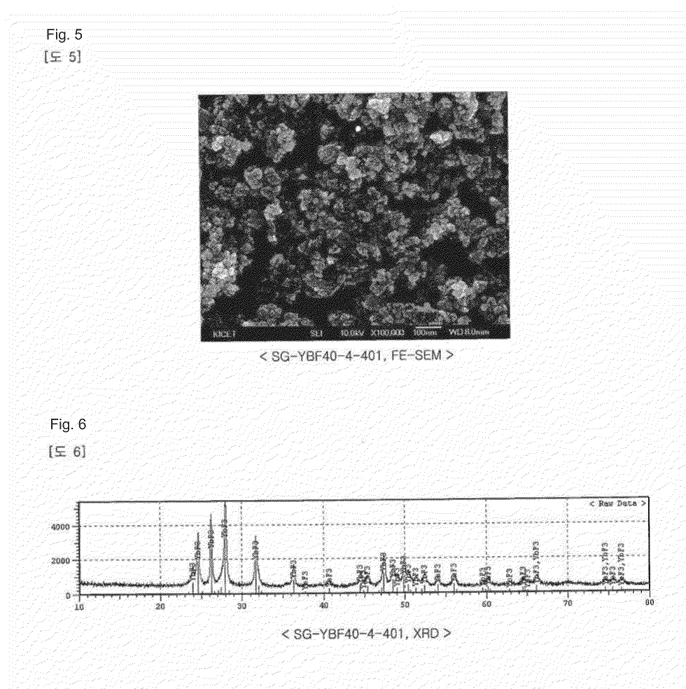

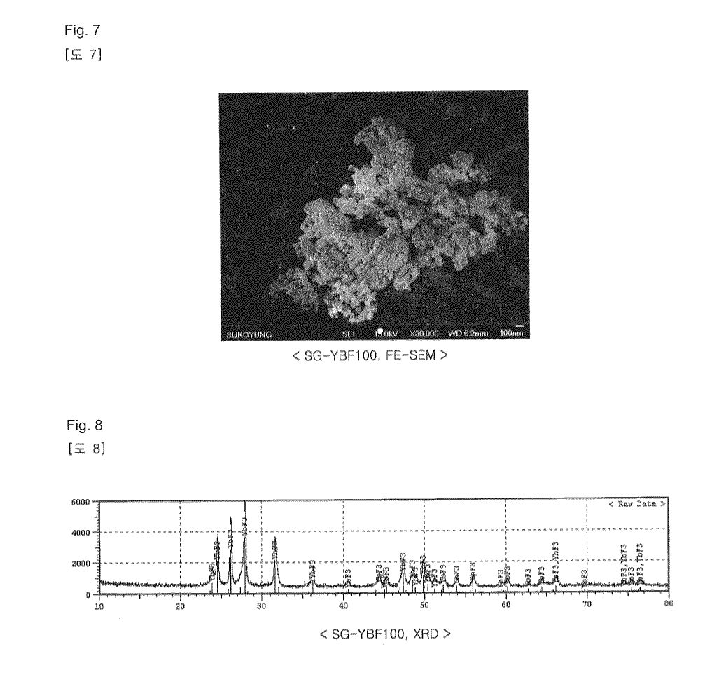

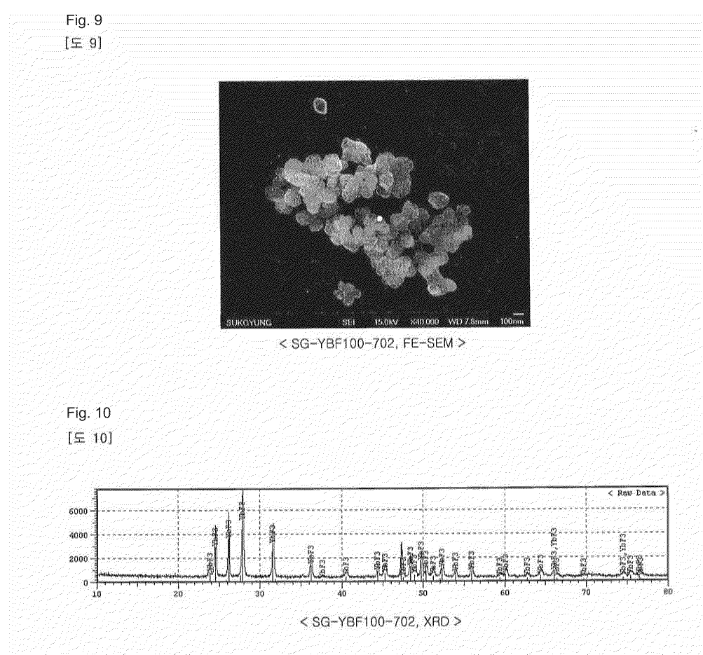

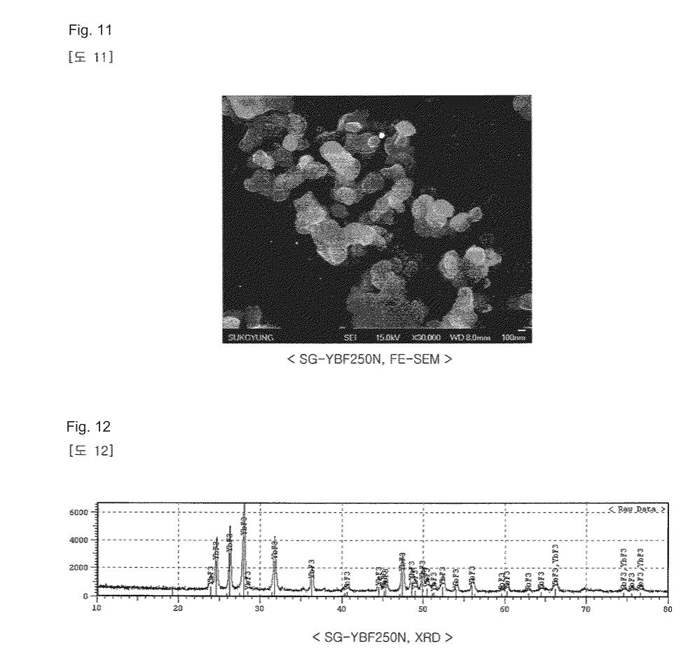

Utilizing Field Emission Scanning Electron Microscopy (FE-SEM), the actual particle sizes of the ytterbium fluoride manufactured in accordance with the invention are shown in FIGS. 5, 7, 9, and 11. The specific surface area of the ytterbium fluoride was determined to be about 11 to 18 m²/g and the refractive index was measured to be 1.53. Hereinafter, ytterbium fluoride is referenced as a radiopaque agent powder.

The radiopaque agent powder is then blended with a plastic material, wherein a preferred composition comprises a fill ratio of about 30 parts nanoparticles of ytterbium fluoride, about 70 parts of plastic material, and about 0.1 parts of plasticizer, dispersant additives. A resin of PVC is the preferred suitable plastic material. Preferably, the composition contains about one to about 50 weight % of the plastic material. The radiopaque agent powder, selected for its uniform particle shape and controlled particle size distribution as described above, is subsequently introduced into a vat containing the molten plastic material. Thus, the solid powder, molten polymer and additives are homogenized as they are agitated and subsequently introduced into the melt stream via the extrusion process.

The resulting polymer/nanoparticle mixtures were then tested for radiopacity and appearance as to translucency, the results being as follows:

Radiopacity: 4.5 mm as Aluminum thickness.

Appearance: Translucent.

As with Example 1, other suitable plastic materials include silicones, polypropylene, polyester, polyolefin, fluoropolymers such as polytetrafluoroethylene (PTFE), polyethyl urethanes, polyether block amides (PEBA), polyethylene terephthalate (PET) and blends or mixtures thereof.

The following three tables set forth the measured radiopacity of three different materials, polypropylene (TABLE 1), silicone (TABLE 2) and PEBEX® (TABLE 3), using various amounts of the noted ytterbium material fillers. PEBEX® is a tradename for polyether block amides (PEBA) manufactured by Arkema Inc. of Philadelphia, Pa. It is a plasticizer-free thermoplastic elastomer belonging to the engineering polymer family. These amides are easy to process by injection molding and profile or film extrusion. PEBEX® can also be easily melt blended with other polymers.

The ytterbium filler materials used in the illustrated examples include 702N (200 nm YbF₃ supplied by Sukgyung AT Co., Inc.), 401 (40 nm YbF₃ supplied by Sukgyung AT Co., Inc.), BAS700 (700 nm Barium Sulfate supplied by Sukgyung AT Co., Inc.), 402 (40 nm YbF₃ supplied by Sukgyung AT Co., Inc.), and BaSO₄ used in amounts of 10%, 20% or 40% by weight. The composition was then formed into a disk, the thickness of the disk measured and recorded, and tested for radiopacity.

TABLE 1
Polypropylene Material

| Filler | Thickness(mm) | Radiopacity |
|---|---|---|
| 10% 702N | 0.90 | <0.6 |
| 20% 702N | 1.02 | 1.3 |
| 10% 401 | 1.01 | <0.6 |
| 20% 401 | 1.00 | 1.1 |
| 10% BAS700 | 0.95 | <0.6 |
| 20% BAS700 | 1.10 | <0.6 |

TABLE 2
Silicone Material

| Filler | Thickness(mm) | Radiopacity |
|---|---|---|
| 10% 702N | 2.10 | 0.4 |
| 20% 702N | 1.53 | 1.3 |
| 10% 401 | 1.72 | 0.9 |
| 20% 401 | 1.65 | 1.3 |
| 10% BAS700 | 1.71 | 0.4 |
| 20% BAS700 | 1.52 | 0.8 |

TABLE 3
PEBAX ® Material

| Filler | Thickness(mm) | Radiopacity |
|---|---|---|
| 20% 702N | 0.68 | 1.1 |
| 40% 702N | 0.74 | 2.9 |
| 20% 402 | 0.66 | 1.9 |
| 40% 402 | 0.67 | 3.7 |
| 20% BaSO₄ | 0.74 | 0.6 |
| 40% BaSO₄ | 0.74 | 1.5 |
| None | 0.69 | 0.2 |

The results clearly illustrate improved radiopacity in all examples using ytterbium filler material, and even greater improvements at higher percentages of the filler material.

In one application of an embodiment in accordance with the present invention, a medical tool is provided, wherein the medical tool comprises an elongated shaft having a proximal end, a distal end and a lumen there between, wherein the distal end comprises a polymer having an amount of radiopaque nanoparticles dispersed therein. One example of the medical tool as contemplated for the purposes of this invention includes a catheter. The polymer further comprises an additive, wherein the polymer is selected from the group of polymers consisting of silicones, polypropylene, polyesters, polyethylene terephthalate (PET), polyolefins, fluoropolymers, polyvinyl chloride (PVC), polyethylene urethanes, polyether block amides (PEBA) and any combination or mixtures thereof. It is contemplated that the radiopaque nanoparticles dispersed in the medical tool comprise a compound selected from the group consisting of ytterbium, an alloy of ytterbium, and a ytterbium composite such as ytterbium trioxide, ytterbium fluoride. The radiopaque nanoparticle to polymer ratio, by weight, in the medical tool is in the range of from about 99:1 to about 50:50.

The medical tool as contemplated in an embodiment, the radiopaque nanoparticles have an average particle size in the range of from about 30 nm to about 2 μm. The radiopaque nanoparticles as contemplated herein, also have an average surface area in the range of from about 30 to about 35^{m2}/g. It is also contemplated that the refractive index measured therein are in the range of from about 1.53 to about 1.58.

The medical tool as contemplated in another embodiment, the radiopaque nanoparticles have an average particle size in the range of from about 10 nm to 500 nm.

In another embodiment in accordance with the present invention, a catheter is provided, wherein the catheter comprises an elongated shaft having a proximal end, a distal end and a lumen there between, wherein the distal end comprises a polymer having an amount of radiopaque nanoparticles dispersed therein. The polymer further comprises an additive, wherein the polymer is selected from the group of polymers consisting of silicones, polypropylene, polyesters, polyethylene terephthalate (PET), polyolefins, fluoropolymers, polyvinyl chloride (PVC), polyethylene urethanes, polyether block amides (PEBA) and any combination or mixtures thereof. It is contemplated that the radiopaque nanoparticles dispersed in the catheter comprise a compound selected from the group consisting of ytterbium, an alloy of ytterbium, and a ytterbium composite such as ytterbium trioxide, ytterbium fluoride. The radiopaque nanoparticle to polymer ratio, by weight, in the catheter is in the range of from about 99:1 to about 50:50.

The catheter as contemplated herein, the radiopaque nanoparticles have an average particle size in the range of from about 30 nm to about 2 μm. The radiopaque nanoparticles as contemplated herein, also have an average surface area in the range of from about 1 to about 18^{m2}/g. It is also contemplated that the refractive index measured therein are in the range of from about 1.45 to about 1.55.

The catheter as contemplated in another embodiment, the radiopaque nanoparticles have an average particle size in the range of from about 10 nm to 500 nm.

It should be emphasized that the above-described embodiments of the present invention, particularly, any “preferred” embodiments, are possible examples of implementations merely set forth for a clear understanding of the principles for the invention. Many variations and modifications may be made to the above-described embodiment(s) of the invention without substantially departing from the spirit and principles of the invention. All such modifications are intended to be included herein within the scope of this disclosure and the present invention, and protected by the following claims.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A medical tool comprising an elongated shaft having a proximal end, a distal end and a lumen there between, wherein the distal end comprises a polymer having an amount of radiopaque nanoparticles dispersed therein.

2. The medical tool of claim 1, wherein the radiopaque nanoparticles comprise a compound selected from the group consisting of ytterbium, an alloy of ytterbium, and a ytterbium composite.

3. The medical tool of claim 1, wherein the radiopaque nanoparticles comprise ytterbium trioxide.

4. The medical tool of claim 1, wherein the radiopaque nanoparticles comprise ytterbium fluoride.

5. The medical tool of claim 1, wherein the radiopaque nanoparticles have an average particle size in the range of from about 30 to about 130 nm.

6. The medical tool of claim 1, wherein the radiopaque nanoparticles have an average particle size in the range of from about 10 to about 500 nm.

7. The medical tool of claim 2, wherein the radiopaque nanoparticles have an average particle size in the range of from about 30 to about 130 nm.

8. The medical tool of claim 2, wherein the radiopaque nanoparticles have an average particle size in the range of from about 10 to about 500 nm.

9. The medical tool of claim 1, wherein the radiopaque nanoparticles have an average surface area in the range of from about 16 to about 18 m²/g.

10. The medical tool of claim 1, wherein the polymer having the radiopaque nanoparticles dispersed therein has a refractive index in the range of from about 1.53 to about 1.58.

11. The medical tool of claim 1, wherein the polymer further comprises an additive.

12. The medical tool of claim 1, wherein the polymer is selected from a group of polymers consisting of silicones, polypropylene, polyesters, polyethylene terephthalate (PET), polyolefins, fluoropolymers, polyvinyl chloride (PVC), polyethylene urethanes, polyether block amides (PEBA) and any combination or mixtures thereof.

13-26. (canceled)

27. A radiopaque material comprising:
a polymer;
nanoparticles of at least one of ytterbium, an alloy of ytterbium, a ytterbium composite;
wherein the ratio of polymer to nanoparticles, by weight, is in the range of from about 1:99 to about 50:50.

28-40. (canceled)

41. A method of forming a medical device comprising the steps of:
providing an amount of each:
radiopaque nanoparticles, and
a polymer having a melting point;
heating the polymer to a temperature above the melting point to create a polymer melt;
adding the amount of radiopaque nanoparticles to the polymer melt to create a radiopaque polymer material;
mixing the radiopaque polymer material to create a homogenous polymer;
forming the homogenous polymer into a catheter component, and
cooling the homogenous polymer.

42. The method of claim 41, further comprising the step adding an additive to the polymer melt.

43. The method of claim 41, wherein the ratio of polymer to radiopaque nanoparticles, by weight, is in the range of from about 1:99 to about 50:50.

44-45. (canceled)

46. The method of claim 41, wherein the nanoparticles have an average particle size in the range of from about 30 to about 130 nm.

47. The method of claim 41, wherein the nanoparticles have an average particle size in the range of from about 10 to about 500 nm.

48. The method of claim 41, wherein the nanoparticles have an average surface area in the range of from about 16 to about 18 m²/g.

49. The method of claim 41, wherein the material has a refractive index in the range of from about 1.53 to about 1.58.
